(19) United States
(12) Patent Application Publication
(10) Pub. No.: US 2010/0255082 A1
(43) Pub. Date: Oct. 7, 2010

(54) Functionally Coated Breakable Tablets

(76) Inventors: Ishwar Chauhan, Voorhees, NJ (US); Siva Rama Krishna Nutalapati, Princeton, NJ (US)

Correspondence Address: LICATA & TYRRELL P.C., 66 E. MAIN STREET, MARLTON, NJ, 08053, US

(21) Appl. No.: 12/742,162

(22) PCT Filed: Jul. 24, 2009
(86) PCT No.: PCT/US2009/051620

§ 371 Date: May 10, 2010

Related U.S. Application Data
(60) Provisional application No. 61/085,085, filed on Jul. 31, 2008.

(51) Int. Cl. A61K 9/48; A61K 9/44

(52) U.S. Cl. 424/451; 424/467

(57) ABSTRACT

Tablet and capsule formulations with a similar in vitro drug release profile for whole tablet and when broken and/or a bioequivalent drug release profile when taken whole or when broken are provided. In addition, coated splittable tablets are provided. Methods for production of these formulations and tablets and their administration are also provided.

This patent application claims the benefit of priority from U.S. Provisional Application Ser. No. 61/085,085, filed Jul. 31, 2008, teachings of which are herein incorporated by reference in their entirety.

### FIELD OF THE INVENTION

The present invention provides oral tablet and capsule formulations with at least two drug containing units separated by a plug. These oral tablet and capsule formulations provide a similar in vitro drug release profile for whole tablet and when broken and/or a bioequivalent drug release profile when taken whole or when broken or split into portions for easier swallowing. These formulations also provide a means for effectively splitting coated tablets. Methods for production and use of these oral tablet and capsule formulations are also provided.

### BACKGROUND OF THE INVENTION

People often need to break or sprinkle their medications if they cannot swallow the entire tablet or capsule. Most often the entire tablet or capsule of medication is sprinkled over a teaspoonful of soft food such as apple sauce, custard, ice cream, oatmeal, pudding or yoghurt. The entire spoonful of the sprinkle/food mixture is then swallowed without chewing. It may be helpful to have the patient drink fluids immediately to make sure all of the mixture is swallowed. For a medicine to be approved by the Food and Drug Administration for administration via this manner, the drug release profile upon sprinkling must be bioequivalent to the drug release profile of the whole tablet or capsule.

Tablet-splitting has also been suggested to help make medications more affordable for uninsured or financially strained people and/or when there is a need for dose titration and tablets in low enough doses are not available. See, e.g. sunherald with the extension .com/170/story/578247.html of the world wide web; Noviasky et al. The Journal of Family Practice, jfponline with the extension .com/Pages.asp?AID=4326 of the world wide web;

For many tablets, particularly coated controlled, modified or delayed release prescription medication tablets, the drug release profile is altered or compromised upon breaking or splitting. Generally, single unit coated tablets cannot be broken into small pieces without compromising the coating. Such compromises result in differences in drug release profile rendering breaking or sprinkling of the tablet or capsule unacceptable.

Multiparticulate formulations are currently the only FDA approved formulations that can be taken whole or sprinkled.

The American Medical Society and American Pharmacists Association recommends against splitting tablets that are modified release, combination products, unscored, film coated, friable or dose critical (Noviasky et al. The Journal of Family Practice, jfponline with the extension .com/Pages.asp?AID=4326 of the world wide web).

### SUMMARY OF THE INVENTION

The present invention relates to core tablets comprising two or more drug containing units, each unit being separated by a plug having a breakable score.

In one embodiment, the core tablet has a similar in vitro drug release profile for whole tablet and when broken and/or a bioequivalent drug release profile when taken whole or when broken into pieces at the breakable score or scores on the plugs. This core tablet is thus useful in tablet and capsule formulations which are broken and sprinkled over a teaspoonful of soft food such as apple sauce, custard, ice cream, oatmeal, pudding or yoghurt to facilitate swallowing.

In this embodiment of the present invention, the core tablet may be coated or compressed with one or more functional films or coatings. Further, the core tablet may be coated with a subcoating prior to coating or compressing with the functional film or coating.

In this embodiment, one or more core tablets can be encapsulated in a capsule. These core tablets may be coated or compressed with one or more functional films or coatings and encapsulated into a capsule. Further, the core tablet may be coated with a subcoating prior to coating or compressing with the functional film or coating.

Another embodiment of the present invention relates to splittable coated tablet and capsule formulations. In this embodiment, the core tablet is designed to be split or divided at the scores on the plugs into drug containing units administered separately to a patient. In this embodiment, the core tablet is coated or compressed with one or more functional films or coatings. The functionally coated tablet comprises a core tablet with one or more scores and a functional coating on the core tablet, wherein the tablet can be broken into two or more pieces at the one or more scores of the drug containing core tablet without comprising functionality of the coating. This embodiment of the present invention is thus useful in providing coated tablet formulations which can be split to help make medications more affordable for uninsured or financially strained people and/or when there is a need for dose titration and coated tablets in low enough doses are not available.

In this embodiment, a subcoating may be applied to the core tablet prior to coating or compressing with the functional film or coating.

Another aspect of the present invention relates to a method for producing core tablets having a similar in vitro drug release profile for whole tablet and when broken and/or a bioequivalent drug release profile when taken whole or when broken or core tablets which can be coated and then split to help make medications more affordable for uninsured or financially strained people and/or when there is a need for dose titration and coated tablets in low enough doses are not available. The method comprises compressing into a core tablet two or more drug containing units, each unit being separated by a plug and scoring a breakable score into each plug of the core tablet.

The core tablet can then be coated or compressed with one or more functional films or coatings after scoring. The core tablet may be coated with a subcoating prior to coating or compressing with the functional film or coating.

For sprinklable, bioequivalent formulations, one or more of the core or coated tablets can be encapsulated in a capsule to provide a capsule formulation whose contents can be broken into smaller portions for easy swallowing.

Another aspect of the present invention relates to a method for orally administering a drug to a patient which comprises breaking the above described core tablet or coated tablet or opening the above-described capsule and breaking the core tablet or coated tablet into drug containing units at each breakable score on each plug and administering all broken segments to the patient.

Yet another aspect of the present invention relates to a method for orally administering a drug to a patient which comprises breaking from the above described coated tablet a drug containing unit at a breakable score of the plug and administering the drug containing unit to the patient.

### BRIEF DESCRIPTION OF THE FIGURES

FIG. 1 is a diagram of an exemplary core tablet of the present invention comprising a single drug. The drug containing units are designated as “Drug” and are separated by the plugs, designated by “Plug”, with breakable scores.

FIG. 2 is a cross-sectional view of the core tablet of FIG. 1.

FIG. 3 is a diagram of an exemplary core tablet of the present invention comprising drug containing units with multiple drug containing layers and multiple drug release layers. The multiple drug containing layers of each drug containing unit are designated as “Drug 1” and “Drug 2”. The multiple drug release layers of each drug containing unit are designated as immediate release layer “IR” and modified release layer “MR”. Each drug containing unit of the core tablet is separated by a plug, designated by “Plug”.

FIG. 4 is a cross-sectional view of an exemplary tablet of the present invention depicting the core tablet with breakable scores coated with a subcoating and a functional coating or film.

FIG. 5 is a diagram of an exemplary capsule of the present invention showing a core or coated tablet of the present invention encapsulated within a capsule.

### DETAILED DESCRIPTION OF THE INVENTION

The present invention provides new formulations for orally administered tablets and capsules.

In one embodiment, the tablet and capsule formulations of the present invention exhibit a similar in vitro drug release profile for whole tablet and when broken and/or bioequivalent drug release profiles when taken whole or when broken.

In another embodiment, the formulations of the present invention provides a means for effectively splitting coated tablets to help make medications more affordable for uninsured or financially strained people and/or when there is a need for dose titration and coated tablets in low enough doses are not available. In this embodiment, the functionally coated tablet comprises a core tablet with one or more scores and a functional coating on the core tablet, wherein the tablet can be broken into two or more pieces at the one or more scores of the drug containing core tablet without comprising functionality of the coating.

Formulations of the present invention can be used with any orally administered drug which can be compressed into tablet layers.

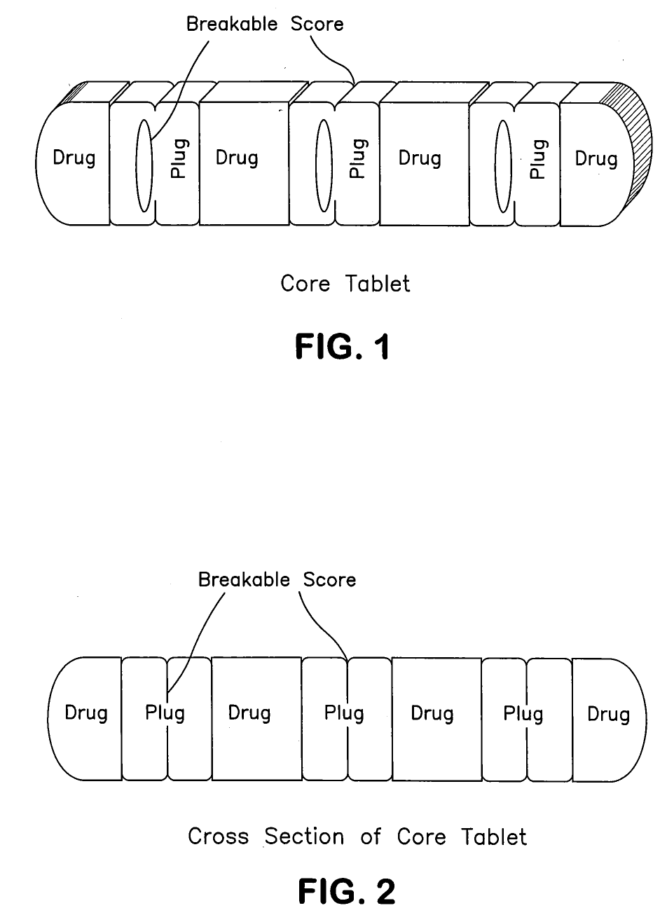

Exemplary embodiments of core tablets and coated tablets of the present invention are depicted in FIGS. 1 through 4.

As shown in FIGS. 1 through 3, a tablet of the present invention comprises a core tablet comprising a two or more drug containing units, designated by “Drug” separated by plugs designated by “Plug”. Each plug has a breakable score line for breakage of the core tablet into multiple pieces. In one embodiment, the pieces are sized to be more easily swallowed by the patient. In another embodiment, the pieces are sized to deliver a smaller fraction of the drug or drugs in the tablet to the patient. As shown in FIG. 4, the core tablet can be coated or compressed with one or more functional coatings or films. Further, before coating or compressing the core tablet with a functional coating or film, the core table may first be coated with an optional subcoating.

The drug containing unit may comprise a single orally administered drug or multiple orally administered drugs. Each drug containing unit may comprise the same amount of drug or drugs. Alternatively, each drug containing unit may contain different amounts of drug or drugs and/or different drugs when more than one drug is being administered.

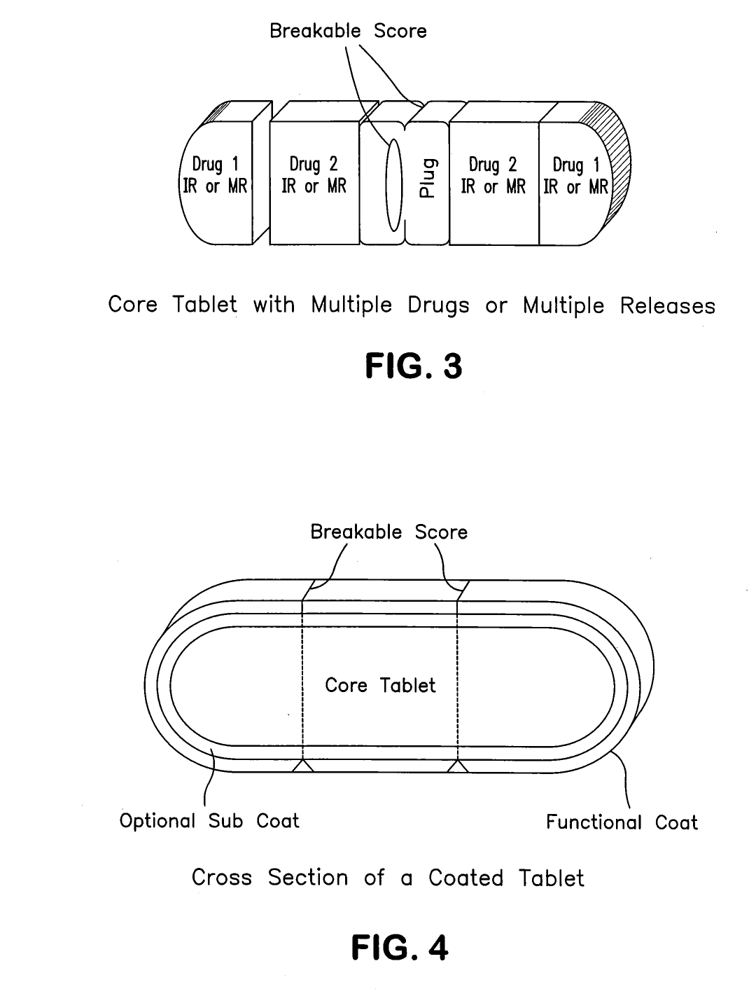

As shown in FIG. 3, the drug containing unit may comprise more than one drug and/or may exhibit different drug release profiles. In FIG. 3, multiple drug containing layers of the drug containing unit are designated as “Drug 1” and “Drug 2” and the different drug release layers of the drug containing unit are designated as immediate release layer “IR” and modified release layer “MR”. As will be understood by the skilled artisan upon reading this disclosure, additional drugs may be included as well as alternative release layers. For example, when the core tablet is comprised of a single drug the possible drug release mechanisms include immediate release, modified release (all types of release mechanisms except immediate release) and a combination of both. When the core tablet is comprised of multiple drugs the possible drug release mechanisms include all drugs exhibiting immediate release, all drugs exhibiting modified release (all types of release mechanisms except immediate release) and/or some drugs exhibiting immediate release and some drugs exhibiting modified release.

Any orally active agent can be included as a drug in the core tablet. Examples include, but are not limited to, alpha-2 adrenergic agents, analgesics, angiotensin-converting enzyme (ACE) inhibitors, antianxiety agents, antiarrhythmics, antibacterials, antibiotics, anticoagulants, anticonvulsants, antidepressants, antidiabetics, antiemetics, antiepileptics, antifungals, antihelminthics, antihistamines, antihyperlipidemics, antihypertensives, antiinfectives, antimalarials, antimicrobials, antimigraine agents, antimuscarinic agents, antineoplastic agents, antiprotozoals, antipsychotics, antispasmodics, antiretroviral agents, antivirals, attention-deficit hyperactivity disorder (ADHD) agents, β-blockers, calcium channel blockers, chemotherapeutic agents, cholinesterase inhibitors, Cox-2 inhibitors, decongestants, diuretics, histamine-2 receptor antagonists, hypnotics, hypoglycemic agents, hypotensive agents, immunosuppresants, lipotropics, neuroleptics, opioid analgesics, peripheral vasodilators/vasoconstrictors, proton pump inhibitors, sedatives, serotonin receptor agonists, sympathomimetics as well as pharmaceutically acceptable salts, solvates, hydrates, stereoisomers (racemates, individual enantiomers or diastereomers, or any combination thereof), or polymorphs thereof, or pharmaceutically acceptable combinations comprising at least one of the foregoing active agents, and the like.

In one embodiment, the immediate release drug layer or layers or portion or portions of the drug containing unit of the core tablet of the present invention is prepared by direct compression of a mixture of the drug or drugs with a suitable carrier or excipient, such as carbohydrate or protein fillers, such as sugars, including lactose, sucrose, mannitol, or sorbitol; starch from corn, wheat, rice, potato, or other plants; cellulose, such as methyl cellulose, hydroxypropylmethyl cellulose, sodium carboxymethylcellulose, or microcrystalline cellulose; gums including arabic and tragacanth; proteins such as gelatin and collagen; inorganics, such as kaolin, calcium carbonate, dicalcium phosphate, sodium chloride; magnesium carbonate; magnesium oxide; and other agents such as acacia and alginic acid.

Agents that facilitate disintegration and/or solubilization can also be added to the drug layer of the drug containing unit. Examples include, but are not limited to cross-linked polyvinyl pyrrolidone, sodium starch glycolate, Croscarmellose Sodium, alginic acid, or a salt thereof, such as sodium alginate, microcrystalline cellulose and corn starch.

Tablet binders can be used in the drug layer of the drug containing unit as well and include, but are not limited to, acacia, methylcellulose, sodium carboxymethylcellulose, polyvinylpyrrolidone (POVIDONE), hydroxypropyl cellulose, hydroxypropyl methylcellulose, sucrose, starch and ethylcellulose.

Lubricants can also be used in the drug layer of the drug containing unit and include, but are not limited to, magnesium stearates, stearic acid, sodium stearyl fumerate, talc, waxes, oils, silicon dioxide and colloidal silica.

The drug layer or layers of the drug containing unit of the tablets are formulated, for example, by preparing a powder mixture of drug or drugs by dry blending or granulating or slugging, adding a lubricant and disintegrant and pressing the mixture into tablet layers.

A modified release layer or layers or portion or portions of a drug containing unit of the core tablet can be prepared by incorporating release retarding excipients into the above-described formulation for the immediate release drug layer or portion, and either completely omitting or reducing the amount of disintegrants.

Examples of release retarding excipients include, but are not limited to hydrophilic polymers such as hydroxypropylmethylcellulose, hydroxymethylcellulose, hydroxypropylcellulose and hydroxyethylcellulose, and which swell in contact with aqueous liquids, and control release of the drug by diffusion through the swollen polymer network.

Examples of other release retarding excipients include, but are not limited to, waxes such as carnauba wax, bees wax stearic acid and gums such as acacia, acrylic polymers, shellac, zein, polyvinylpyrrolidine including crosslinked polyvinylpyrrolidinone, vinyl acetate copolymers, polyethylene oxides, polyvinyl alcohols, and combinations comprising at least one of the foregoing materials.

The modified release layer or layers of the drug containing unit of the tablets are formulated, for example, by preparing the powder mixture of drug or drugs by dry blending or granulating or slugging, adding a lubricant and release retarding excipients pressing the mixture into tablet layers.

The plugs may comprise any biocompatible easily breakable or splittable compound or mixture of compounds. The plugs may be soluble or insoluble, permeable or impermeable, swellable or non swellable, pH dependent or pH independent or any combination thereof depending upon the drug or drugs to be orally administered and/or the release mechanism required. Preferably, the plugs are inert, insoluble and impermeable to drug in the drug containing unit regardless of thickness so that breakage of the core tablet at score in the plug has no impact on drug release. Accordingly, in one embodiment, the plug comprises no drug or drug in an amount which does not significantly modify the in vitro drug release profile and/or bioequivalence and/or functionality of a functional coating or film on the tablet upon breaking or splitting. As will be understood by the skilled artisan upon reading this disclosure, however, drug in an amount which does modify bioequivalence and/or functionality may be included in the plug and such inclusion does not circumvent this invention.

Exemplary biocompatible materials for use in the plugs include, but not limited to, waxes, polymers, gums and other pharmaceutically acceptable excipients either alone or in combination.

Exemplary wax excipients include, but are not limited to, wax and wax-like excipients such as carnauba wax, vegetable wax, fruit wax, microcrystalline wax, bees wax (white or bleached, and yellow), hydrocarbon wax, paraffin wax, cetyl esters wax or a combination comprising at least one of the foregoing waxes. Other suitable wax excipients include, for example, fatty alcohols (such as lauryl, myristyl, stearyl, cetyl or specifically cetostearyl alcohol), hydrogenated vegetable oil, hydrogenated castor oil, fatty acids such as stearic acid, fatty acid esters including fatty acid glycerides (mono-, di-, and tri-glycerides), polyethylene glycol (PEG) having a molecular weight of greater than about 3000 number average molecular weight, Mₙ (e.g. PEG 3350, PEG 4000, PEG 4600, PEG 6000, and PEG 8000), or a combination comprising at least one of the foregoing.

Exemplary polymer excipients include, for example acrylic polymers, alkylcelluloses including substituted alkylcelluloses, shellac, zein, polyvinylpyrrolidine including crosslinked polyvinylpyrrolidinone, vinyl acetate copolymers, polyethylene oxides, polyvinyl alcohols, and combinations comprising at least one of the foregoing materials.

Suitable acrylic polymers that can be used as a plug include, but are not limited to, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, or a combination comprising at least one of the foregoing polymers.

Suitable alkylcelluloses and substituted alkyl celluloses include, but are not limited to, methyl cellulose, ethylcellulose, hydroxy or carboxy substituted alkyl celluloses (e.g., hydroxylpropylcellulose, crosslinked hydroxypropylcellulose, carboxymethylcellulose, crosslinked sodium carboxymethylcellulose), hydroxy substituted alkyl-alkyl celluloses (e.g., hydroxypropylmethylcellulose), or a combination comprising at least one of the foregoing.

Exemplary additional pharmaceutically acceptable excipients for use in the plugs include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, magnesium aluminum silicate (Veegum), and larch arabogalactan); alginates; polyethylene oxide; inorganic calcium salts; silicic acid; and combinations thereof.

Fillers, tablet binders, pH modifiers and lubricants, including the aforementioned, can be used in the plugs singly or in combination.

In one embodiment, the plugs of the core tablet of the present invention are formulated, for example, by preparing a powder mixture of plug material by dry blending or granulating or slugging, adding a lubricant and pressing into tablet layers.

Two or more drug containing units are then compressed together with plugs to form a single core tablet of the present invention.

The core tablet is preferably scored on the surface of each plug in a post-compression process. Such post-tablet scoring can be performed by removal of plug material from the scored area using a cutting device or instrument such as, but not limited to a blade, rasp, file, laser or the like. Optionally, when the functional coat is applied via compression process, the score can be made after compression of the functional coat.

The core tablet can then be coated or compressed with one or more functional coatings or films. By “functional coating or film” it is meant a coating that modifies the release properties of the total formulation. Examples of such coatings or films include, but are not limited to, controlled release, delayed release, modified release, enteric coating, pH dependent coatings, pH independent coatings, and any combinations thereof.

The functional coating material can be in the form of a film coating comprising a solution or dispersion or a compressible powder mixture of a hydrophilic and/or hydrophobic polymer. Solvents used for application of the functional coating include pharmaceutically acceptable solvents, such as water, methanol, ethanol, isopropanol, acetone, methylene chloride, and a combination comprising at least one of the foregoing solvents.

Exemplary functional coating materials include film forming polymers such as acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, an alkylcellulose including methylcellulose or ethylcellulose, a hydroxyalkylcellulose such as hydroxymethylcellulose, hydroxyethylcellulose, hydroxypropylcellulose and hydroxybutylcellulose, a hydroxyalkyl alkylcellulose such as hydroxyethyl methylcellulose and hydroxypropyl methylcellulose, a carboxyalkylcellulose such as carboxymethylcellulose, an alkali metal salt of carboxyalkylcelluloses such as sodium carboxymethylcellulose, a carboxyalkyl alkylcellulose such as carboxymethyl ethylcellulose, a carboxyalkylcellulose ester, a starch, a pectin such as sodium carboxymethylamylopectine, a chitin derivate such as chitosan, a polysaccharide such as alginic acid, alkali metal and ammonium salts thereof, a carrageenan, a galactomannan, traganth, agar-agar, gum arabicum, guar gum and xanthan gum, a polyacrylic acid and the salts thereof, a polyvinylalcohol, a polyvinylpyrrolidone, a copolymer of polyvinylpyrrolidone with vinyl acetate, a polyalkylene oxide such as polyethylene oxide and polypropylene oxide and a copolymer of ethylene oxide and propylene oxide, or a combination comprising at least one of the foregoing.

The functional coating may optionally comprise a plasticizer, an additional film-former, a pore former, or a combination comprising at least one of the foregoing.

The formulations of the present invention can optionally further comprise a subcoating or non-functional coating. By “non-functional coating” it is meant a coating that does not significantly modify the release properties of the total formulation, for example, a cosmetic coating or an interlayer coating used to separate a functional coating from other components of the formulation. A non-functional coating can have some impact on the release of the active agent due to the initial dissolution, hydration, perforation of the coating, etc., but is not considered to cause significant deviation in release from the non-subcoated composition.

Exemplary subcoating materials include, but are not limited to, film forming polymers like hydroxyalkyl celluloses such as hydroxymethyl cellulose, hydroxyethyl cellulose, hydroxypropyl cellulose and hydroxybutylcellulose, hydroxyalkyl alkylcelluloses such as hydroxyethyl methylcellulose and hydroxypropyl methylcellulose, polyvinylalcohols, polyvinylpyrrolidones, copolymers of polyvinylpyrrolidone with vinyl acetate, and combinations thereof.

For the embodiment of the present invention wherein the tablet has a bioequivalent drug release profile when taken whole or when broken, the drug containing units and plugs of the tablet of the present invention are sized so that upon breakage of the tablet at the breakable scores, the broken pieces of the tablet can be more easily swallowed. Breakage of the tablet into pieces at the breakable scores does not result in changes in the drug release profiles of the tablet either in vivo or in vitro as compared to the tablet as a whole.

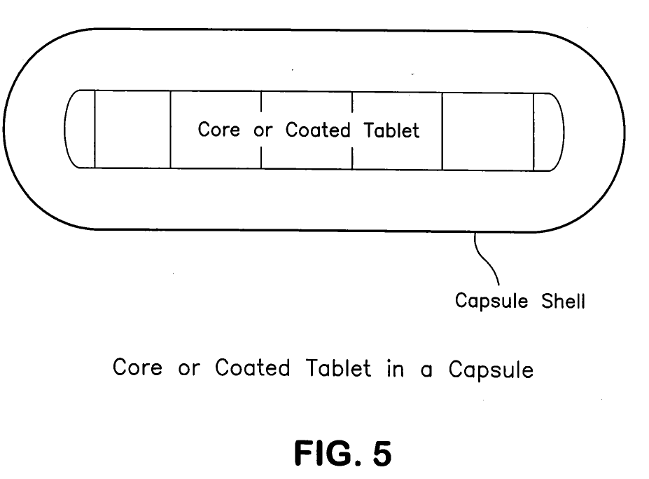

For capsule formulations of the present invention, one or more of the core tablets or coated tablets are encapsulated within a capsule. An exemplary embodiment of a capsule of the present invention is depicted in FIG. 5. Prior to administration to a patient with difficulty swallowing, the capsule is opened; the tablet or tablets are removed and broken into pieces at the breakable scores.

For the embodiment of the present invention wherein the coated tablet is splittable to help make medications more affordable for uninsured or financially strained people and/or when there is a need for dose titration and coated tablets in low enough doses are not available, the drug containing units and plugs are sized for ease in breaking and administering to a patient a single drug containing unit of the tablet.

Also provided in the present invention are methods for producing tablet and capsule formulations which exhibit bioequivalent drug release profiles when taken whole and when broken and coated splittable tablets.

In one embodiment, the method comprises compressing into a core tablet a plurality of drug containing units, each unit separated by a plug. Breakable scores are then made in each plug of the core tablet. In one embodiment, the core tablet is then coated or compressed with one or more functional films or coatings. In this embodiment, the core tablet may be optionally coated with a subcoating prior to coating or compressing with the functional film or coating. In another embodiment, the core tablet is encapsulated in a capsule. In yet another embodiment, the core tablet is coated or compressed with a functional film or coating and then encapsulated into a capsule. In this embodiment, the core tablet may be optionally coated with a subcoating prior to coating or compressing with the functional film or coating.

The tablets and capsules of the present invention are useful for oral administration of drugs to patients with difficulty in swallowing whole tablets or capsules. For this use, the above described core tablet or coated tablet is broken into segments at each breakable score. All broken segments of the core or coated tablet are then administered orally to the patient. For capsule formulation, the core tablet or coated tablet is first removed from its capsule.

These tablets then broken into segments at each breakable score. Again, each of the broken segments is orally administered to the patient.

The present invention also provides functionally coated tablets comprising a core tablet with one or more scores and a functional coating on the core tablet, wherein the tablet can be broken into two or more pieces at the one or more scores of the drug containing core tablet without compromising functionality of the coating. By the phrase “without compromising functionality of the coating” it is meant that the function of the coating remains unchanged upon breaking or splitting of the tablet at the scores. Thus, if the functional coating provides for delayed drug release, timing of release of the drug from the tablet remains the same for the tablet after breaking. Such functionally coated tablets provide for the first time a means for efficiently and effectively splitting tablets of drugs requiring a functional coating or film for effective oral administration. In this embodiment, it is preferred that each drug containing unit comprise the same amount of drug or drugs or drug release layers so that the tablet can be divided and a portion or portions of the tablet can be administered to the patient. Splitting of tablets in this manner helps to make medications more affordable for uninsured or financially strained people and/or provides a means for dose titration and administration of lower doses of coated tablets than are routinely available.

The following nonlimiting examples are provided to further illustrate the present invention.

### EXAMPLES

### Example 1

### Preparation of Enteric-Coated Multilayer Tablets of Omeprazole

The drug layer or portion contained Omeprazole magnesium (22.45 mg/tablet), microcrystalline cellulose (64 mg/tablet), lactose anhydrous (190.55 mg/tablet), hydroxypropyl cellulose (10.00 mg/tablet), croscarmellose sodium (10.00 mg/tablet), and magnesium stearate (3.00 mg/tablet).

The plug layer or portion contained Carnauba wax (128.00 mg/tablet), Dibasic Calcium phosphate (50.00 mg/tablet), Stearic acid (20.00 mg/tablet) and magnesium stearate (2.00 mg/tablet).

The subcoating contained Hydroxypropyl methyl cellulose (12.50 mg/tablet), Polyethylene glycol 400 (2.50 mg/tablet) and purified water which was removed during processing.

The enteric coating contained Eudragit L30D55 (24.32 mg/tablet), triethyl citrate (2.66 mg/tablet), talc (14.62 mg/tablet) and purified water which was removed during processing.

The Drug Containing Unit was Prepared as Follows:

Omeprazole magnesium was dry blended with all the ingredients except magnesium stearate for five minutes in a blender. Magnesium stearate was screened and then added to the blender. The mixture was then blended for another 2 minutes.

The Plug was Prepared as Follows:

Carnauba wax and Dicalcium phosphate were mixed in a collette and granulated with a solution of Stearic acid in ethyl alcohol. The granulate was then dried, milled and transferred to a blender. Magnesium stearate was screened and then added to the blender. The mixture was then blended for another 2 minutes.

The blends were then compressed into a multi-layer tablet with alternate drug containing units and plugs of 100 mg each using a multi-layer tablet press. Core tablets were then scored and subcoated.

The subcoating was prepared by dissolving Hydroxypropyl methyl cellulose and Polyethylene glycol 400 in purified water and sprayed as a coating solution onto the multi layer core tablet bed in a coating pan.

The enteric coating was prepared by mixing Eudragit L30D55 and triethyl citrate in a container using a mixer. In a separate container purified water was mixed with talc using mixer until the talc is evenly dispersed in the water. The talc suspension was then added to the Eudragit dispersion and mixed for 15 minutes. The resulting dispersion was mixed during the entire coating process. Using the coating pan, the Eudragit/Talc dispersion was sprayed onto the subcoated tablets until the required weight gain was achieved.

### Example 2

### Preparation of Extended Release Multilayer Tablets of Fluvastatin Sodium

The drug containing units contained Fluvastatin Sodium (42.12 mg), microcrystalline cellulose (16.88 mg), hypromellose (40.00 mg), and magnesium stearate (1.00 mg).

The plug contained carnauba wax (64 mg), dibasic calcium phosphate (25 mg), stearic acid (10 mg) and magnesium stearate (1 mg).

The subcoating contained hydroxypropyl methyl cellulose (12.50 mg/tablet), polyethylene glycol 400 (2.50 mg/tablet) and purified water which was removed during processing.

The extended release coating contained Surelease (30.90 mg/tablet), hydroxypropyl methyl cellulose (20.60 mg/tablet) and purified water which was removed during processing.

The Drug Containing Unit was Prepared as Follows:

Fluvastatin sodium was dry blended with all the ingredients except magnesium stearate and granulated with purified water. The granulate was dried and milled through a suitable screen. Magnesium stearate was screened and then added to the milled granules. The mixture was then blended for about 2 minutes.

The Plug was Prepared as Follows:

Carnauba wax and dicalcium phosphate were mixed in a collette and granulated with a solution of stearic acid in ethyl alcohol. The granulate was then dried, milled and transferred to a blender. Magnesium stearate was screened and then added to the blender. The mixture was then blended for another 2 minutes.

The blends were then compressed into a multi-layer core tablet in the following sequence: drug containing unit-plug-drug containing unit of 100 mg each using a multi-layer tablet press. Core tablets were then scored and subcoated.

The subcoating was prepared by dissolving hydroxypropyl methyl cellulose and polyethylene glycol 400 in purified water and sprayed as a coating solution onto the multi-layer core tablet in a coating pan.

The Extended Release Coating was Prepared as Follows:

In a container purified water was mixed with hydroxypropyl methyl cellulose using mixer until the hydroxypropyl methyl cellulose was completely dissolved. The hydroxypropyl methyl cellulose solution was then added to the Surelease dispersion and mixed for 15 minutes. The resulting dispersion was mixed during the entire coating process. Using the coating pan, the Surelease/hydroxypropyl methyl cellulose dispersion was sprayed onto the subcoated tablets until the required weight gain was achieved.

1. A functionally coated tablet comprising a core tablet with two or more drug containing units, each unit being separated by a plug having a breakable score, and a functional coating or film compressed with or coated on said core tablet, said functionally coated tablet having a similar in vitro drug release profile for whole tablet and when broken and/or a bioequivalent drug release profile when taken whole or when broken.

2. The functionally coated tablet of claim 1 wherein the drug containing units comprise a single drug.

3. The functionally coated tablet of claim 1 wherein the drug containing units comprise two or more drugs.

4. The functionally coated tablet of claim 1 wherein each drug containing unit comprises one or more different drugs.

5. The functionally coated tablet of claim 1 wherein the drug containing units comprise two or more different drug release layers.

6. The functionally coated tablet of claim 1 further comprising a subcoating between the core tablet and the functional film or coating.

7. The functionally coated tablet of claim 1 wherein the plug comprises no drug or drug in an amount which does not significantly modify the in vitro drug release profile and/or bioequivalence and/or functionality of the functional coating or film on the tablet upon breaking or splitting.

8. A capsule comprising one or more functionally coated tablets of claim 1 encapsulated in a capsule.

9. A method for producing a functionally coated tablet having a similar in vitro drug release profile for whole tablet and when broken and/or a bioequivalent drug release profile when taken whole or when broken, said method comprising compressing into a core tablet two or more drug containing units, each drug containing unit being separated by a plug; scoring a breakable score into each plug of the core tablet; and coating or compressing the core tablet with one or more functional films or coatings.

10. The method of claim 9 further comprising coating the core tablet with a subcoating prior to coating or compressing the core tablet with one or more functional films or coatings.

11. A method for producing a capsule having a similar in vitro drug release profile for whole tablet and when broken and/or a bioequivalent drug release profiles when taken whole or contents of the capsule are broken, said method comprising encapsulating one or more tablets produced in the method of claim 9 in a capsule.

12. A method for orally administering a drug to a patient comprising breaking the functionally coated tablet of claim 1 into segments at each breakable score and administering all broken segments to the patient.

13. A method for orally administering a drug to a patient comprising opening the capsule of claim 8, breaking the functionally coated tablet into segments at each breakable score, and administering all broken segments to the patient.

14. A functionally coated tablet comprising a drug containing core tablet with one or more scores and a functional coat or film on said core tablet, wherein said tablet can be broken into two or more pieces at the one or more scores of the drug containing core tablet without compromising functionality of the coating.

15. The functionally coated tablet of claim 14 wherein the core tablet comprises two or more drug containing units and one or more plugs separating the drug containing units and wherein the scores are placed on the plugs.

16. The functionally coated tablet of claim 14 wherein each drug containing unit of the tablet comprises the same amount of drug or drugs and/or the same drug release layer or layers.

17. The functionally coated tablet of claim 15 wherein the plug comprises no drug or drug in an amount which does not significantly modify the in vitro drug release profile and/or bioequivalence and/or functionality of the functional coating or film on the tablet upon breaking or splitting.
